(19) United States
(12) Patent Application Publication
(10) Pub. No.: US 2012/0291779 A1
(43) Pub. Date: Nov. 22, 2012

(54) FLOW SENSOR AND AEROSOL DELIVERY DEVICE

(75) Inventors: Jacob Roger Haartsen, Eindhoven (NL); Jonathan Stanley Harold Denyer, Chichester (GB); Michael James Robbert Leppard, Hunston (GB); Anthony Dyche, Hayling Island (GB); Ronald Dekker, Valkenswaard (NL); Bout Marcelis, Eindhoven (NL)

(73) Assignee: KONINKLIJKE PHILIPS ELECTRONICS, N.V., Eindhoven (NL)

(21) Appl. No.: 13/522,798

(22) PCT Filed: Dec. 16, 2010
(86) PCT No.: PCT/IB2010/055893

§ 371 Date: Jul. 18, 2012

Related U.S. Application Data
(60) Provisional application No. 61/296,678, filed on Jan. 20, 2010.

(51) Int. Cl. A61M 15/00

(52) U.S. Cl. 128/203.12

(57) ABSTRACT

An aerosol delivery system (e.g., MDI or nebulizer for delivering aerosolized medication to a patient) includes a temperature sensor in an aerosol output pathway of the system. A controller determines that an aerosol generator of the system has released aerosol when the sensor senses a predetermined temperature change in the pathway. The temperature sensor may also comprise a thermal flow sensor that includes a heater and upstream and downstream temperature sensors. The controller compares the upstream and downstream temperatures to determine the presence, direction, and/or magnitude of fluid flow in the pathway. The controller may use the aerosol detection and/or flow detection to monitor compliance with desired use of the system and/or provide real-time instructions to a user for proper use of the system. The controller may record the aerosolization and flow data for later analysis.

The present invention relates generally to sensing the presence of aerosol and/or fluid flow through a pathway of an aerosol delivery system (e.g., metered-dose inhalers (MDIs) and nebulizers) used to deliver an aerosol to, for example, the airways of a patient.

Respiratory diseases such as cystic fibrosis, asthma and COPD are often treated by the delivery of medication in the form of an aerosol (fine mist) directly to the breathing system. This aerosolized medication delivery is commonly facilitated by aerosol delivery systems such as metered-dose inhalers (MDIs) and nebulizers.

MDIs typically include an actuator/aerosol generator and a pressurized canister that contains one or more drug substances, a propellant and often a stabilizing excipient. The formulation is aerosolized through a valve fitted with the actuator. One canister may contain up to several hundred metered doses or more of the drug substance(s). Depending on the medication, each actuation may contain from a few micrograms up to milligrams of the active ingredients delivered in a volume typically between 25 and 100 microliters. To improve ease-of-use and effectiveness of the MDI, a spacer may be added through which the aerosol cloud passes to reach the patient. Operation of MDIs typically involves three steps. First, the MDI is shaken to mix the drug with the propellant and the excipient. Next, a bolus is released into the spacer by pressing the canister. In the third step the drug is inhaled.

A nebulizer typically comprises a mouthpiece, an air in/outlet, an aerosol generator and a liquid container which contains the liquid drug formulation. Additionally, it may comprise a pressure or flow sensor to detect the breathing pattern. As an example, in Respironics' I-neb nebulizer, the aerosol is generated by a piston that vibrates at a high frequency (ultrasonic), which pushes the drug formulation through a mesh. In the I-neb the aerosol generation is not continuous but is adapted to the breathing pattern based on information provided by the pressure sensor. This is to optimize the treatment and avoid spoiling of the medication. The treatment is typically finished after the container has run dry.

One or more embodiments of the present invention provides a thermal flow sensor that includes a base defining upstream and downstream directions; a heater disposed on the base; a first temperature sensor positioned so as to sense a first temperature at a first location; and a downstream temperature sensor disposed on the base downstream of the heater so as to sense a downstream temperature of the base downstream from the heater. The temperature sensors and heater are located relative to each other such that fluid flow past the base in the downstream direction increases a temperature differential between the first temperature and the downstream temperature.

According to one or more of these embodiments, a magnitude of the temperature differential is proportional to a magnitude of the flow rate of the fluid past the base.

According to one or more of these embodiments, the first temperature sensor includes an upstream temperature sensor that is disposed on the base upstream of the heater so as to sense an upstream temperature of the base upstream from the heater.

According to one or more of these embodiments, the temperature differential is the downstream temperature minus the upstream temperature, the temperature differential is positive when fluid is flowing in one of the upstream and downstream directions past the base, and the temperature differential is negative when fluid is flowing in the other of the upstream and downstream directions past the base.

According to one or more of these embodiments, an upstream distance between the upstream temperature sensor and heater is substantially equal to a downstream distance between the downstream temperature sensor and the heater.

According to one or more of these embodiments, the upstream and downstream temperature sensors are positioned such that when the heater is turned on and there is no fluid flow over the base, the upstream and downstream temperatures are substantially identical.

According to one or more of these embodiments, the temperature sensors and heater are located relative to each other such that fluid flow in a downstream direction past the base increases the downstream temperature relative to the upstream temperature.

According to one or more of these embodiments, the temperature sensors and heater are located relative to each other such that fluid flow past the base in an upstream direction increases the upstream temperature relative to the downstream temperature.

According to one or more of these embodiments, the base includes a frame and a membrane connected to the frame, the frame has a higher thermal capacitance than the membrane, the heater is disposed on the membrane, the downstream temperature sensor is positioned to sense a temperature of the membrane downstream from the heater, and the upstream temperature sensor is positioned to sense a temperature of the membrane upstream from the heater.

According to one or more of these embodiments, the base includes a silicon frame and a membrane connected to the silicon frame, the silicon frame has a higher thermal capacitance than the membrane, the heater is disposed on the membrane, the downstream temperature sensor includes a thermocouple having a reference junction disposed on the silicon frame and a sensing junction disposed on the membrane downstream from the heater, and the upstream temperature sensor includes a thermocouple having a reference junction disposed on the silicon frame and a sensing junction disposed on the membrane upstream from the heater.

According to one or more of these embodiments, the sensor is used in combination with an aerosol delivery system that includes an aerosol generator; an aerosol output opening; and a fluid pathway extending from the aerosol generator to the aerosol output opening. The thermal flow sensor is in thermal communication with the pathway. The downstream direction of the base is directed along the fluid pathway toward the aerosol output opening. The system also includes a controller connected to the upstream and downstream temperature sensors to receive from the sensors upstream and downstream temperature signals, respectively, that correlate to the upstream and downstream temperatures, respectively. The controller is constructed and arranged to detect fluid flow within the pathway by comparing the upstream and downstream temperature signals.

According to one or more of these embodiments, the controller is constructed and arranged to determine a direction of fluid flow within the pathway by comparing the upstream and downstream temperature signals.

According to one or more of these embodiments, the controller is constructed and arranged to use a temperature sensor signal from the sensor to detect the presence of aerosol in the fluid pathway.

According to one or more of these embodiments, the controller is constructed and arranged to determine that aerosol is present in the fluid pathway when the temperature sensor signal indicates a temperature below a predetermined temperature threshold.

According to one or more of these embodiments, the predetermined temperature threshold is colder than a predetermined minimum sensed temperature in the absence of aerosol and at a predetermined maximum flow rate.

According to one or more of these embodiments, the controller is constructed and arranged to vary the predetermined temperature threshold as a function of sensed fluid flow rate.

One or more embodiments of the present invention provides a method for detecting fluid flow past a flow sensor. The flow sensor includes a base defining upstream and downstream directions, a heater disposed on the base, a first temperature sensor positioned so as to sense a first temperature at a first location, and a downstream temperature sensor disposed on the base downstream of the heater so as to sense a downstream temperature of the base downstream from the heater. The method includes causing the heater to generate heat; detecting, via the first temperature sensor, a first temperature at a first location on the base; detecting, via the downstream temperature sensor, a downstream temperature of the base downstream from the heater; and determining whether fluid is flowing past the flow sensor by determining whether a temperature differential between the first temperature and the downstream temperature increases.

According to one or more of these embodiments, the method includes recording in a memory the determination of whether fluid is flowing past the flow sensor.

According to one or more of these embodiments, determining whether a temperature differential between the first temperature and the downstream temperature increases includes subtracting a temperature signal from one of the first temperature sensor and the downstream temperature sensor from the other of the first temperature sensor and the downstream temperature sensor.

According to one or more of these embodiments, determining whether a temperature differential between the first temperature and the downstream temperature increases includes dividing a temperature signal from one of the first temperature sensor and the downstream temperature sensor by the other of the first temperature sensor and the downstream temperature sensor.

According to one or more of these embodiments, the temperature differential is sensed in terms of a unit of measurement that is correlated to a temperature difference between the upstream and downstream temperatures.

According to one or more of these embodiments, the method further includes determining from a magnitude of the temperature differential a magnitude of the flow rate of the fluid past the base.

According to one or more of these embodiments, the method further includes determining a direction of flow past the flow sensor based on the temperature differential.

According to one or more of these embodiments, the first temperature sensor includes an upstream temperature sensor that is disposed on the base upstream of the heater so as to sense an upstream temperature of the base upstream from the heater.

According to one or more of these embodiments, the method further includes determining a direction of flow past the flow sensor by comparing the upstream and downstream temperature signals, the direction of flow being based on a sign of the temperature differential.

According to one or more of these embodiments, the upstream and downstream temperature sensors are positioned such that when the heater is turned on and there is no fluid flow over the base, the upstream and downstream temperatures are substantially identical.

According to one or more of these embodiments, the base includes a frame and a membrane connected to the frame, the frame has a higher thermal capacitance than the membrane, the heater is disposed on the membrane, the downstream temperature sensor is positioned to sense a temperature of the membrane downstream from the heater, and the upstream temperature sensor is positioned to sense a temperature of the membrane upstream from the heater.

According to one or more of these embodiments, the base includes a silicon frame and a membrane connected to the silicon frame, the silicon frame has a higher thermal capacitance than the membrane, the heater is disposed on the membrane, the downstream temperature sensor includes a thermocouple having a reference junction disposed on the silicon frame and a sensing junction disposed on the membrane downstream from the heater, and the upstream temperature sensor includes a thermocouple having a reference junction disposed on the silicon frame and a sensing junction disposed on the membrane upstream from the heater.

According to one or more of these embodiments, the sensor is in thermal communication with a fluid pathway of an aerosol delivery system, the aerosol delivery system includes an aerosol generator, and an aerosol output opening, the fluid pathway extends from the aerosol generator to the aerosol output opening, and the downstream direction of the base is directed along the fluid pathway toward the aerosol output opening.

These and other aspects of various embodiments of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. In one embodiment of the invention, the structural components illustrated herein are drawn to scale. It is to be expressly understood, however, that the drawings are for the purpose of illustration and description only and are not intended as a definition of the limits of the invention. In addition, it should be appreciated that structural features shown or described in any one embodiment herein can be used in other embodiments as well. As used in the specification and in the claims, the singular form of “a”, “an”, and “the” include plural referents unless the context clearly dictates otherwise.

For a better understanding of embodiments of the present invention as well as other objects and further features thereof, reference is made to the following description which is to be used in conjunction with the accompanying drawings, where:

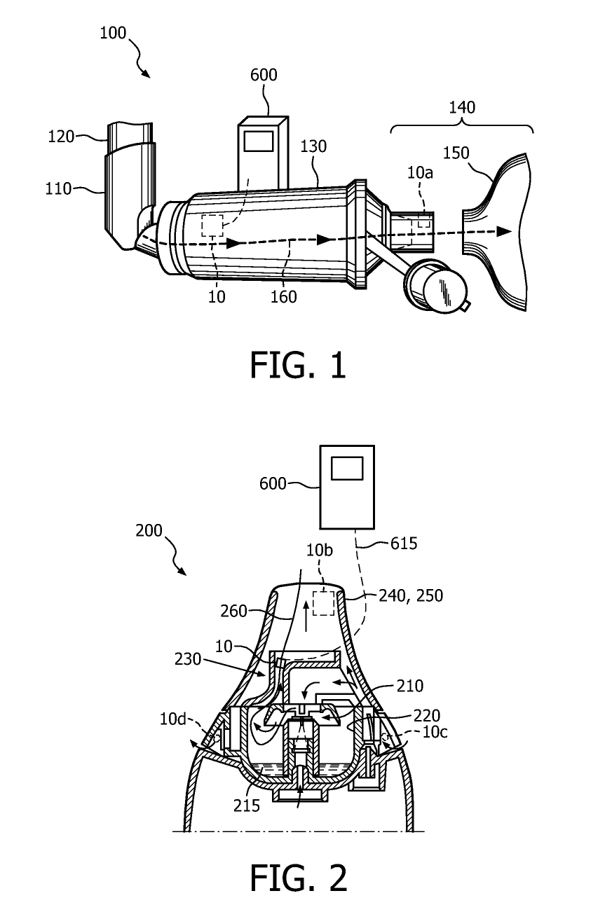

FIG. 1 is a side view of an MDI according to an embodiment of the present invention;

FIG. 2 is a partial cross-sectional view of a jet nebulizer according to an alternative embodiment of the present invention;

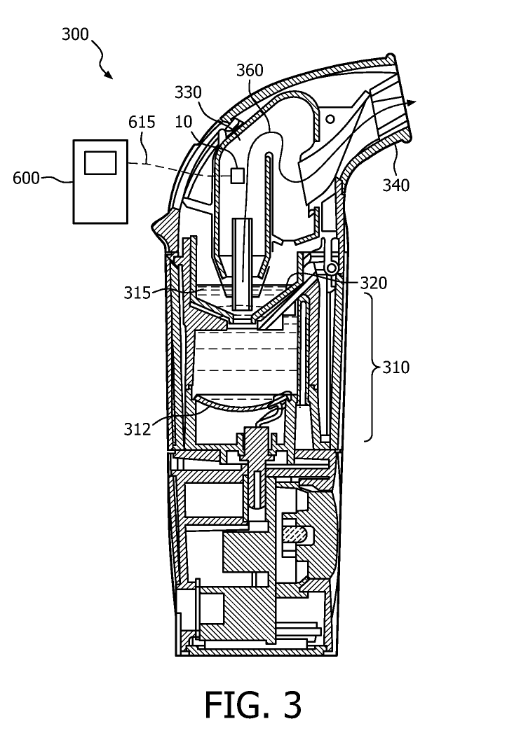

FIG. 3 is a cross-sectional view of an ultrasonic nebulizer according to an alternative embodiment of the present invention;

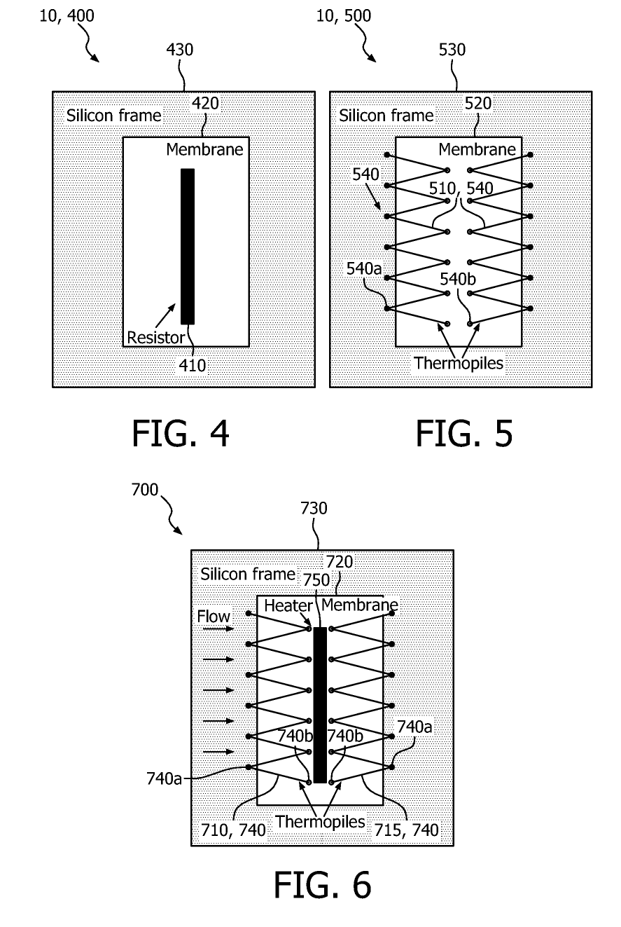

FIG. 4 is a front view of a temperature sensor that may be used in connection with any of the devices shown in FIGS. 1-3 according to various embodiments of the present invention;

FIG. 5 is a front view of an alternative temperature sensor that may be used in connection with any of the devices shown in FIGS. 1-3 according to various embodiments of the present invention;

FIG. 6 is a front view of a thermal flow sensor that may be used in connection with any of the devices shown in FIGS. 1-3 according to various embodiments of the present invention;

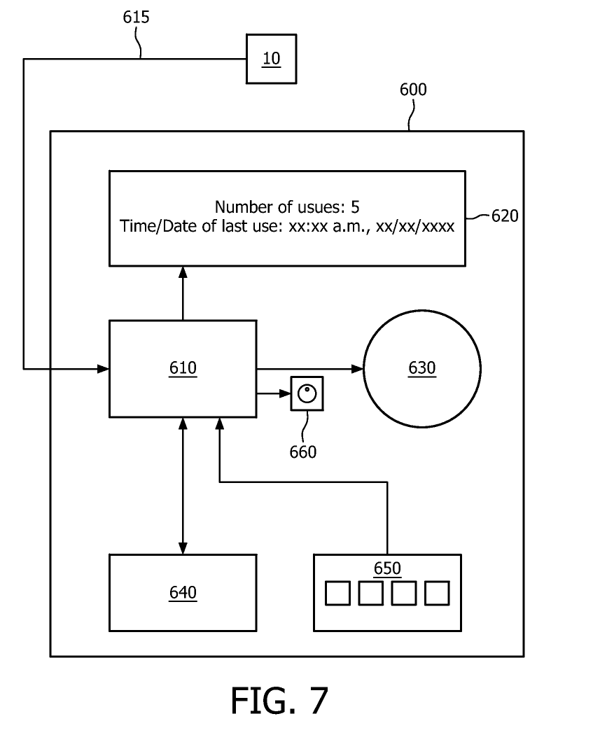

FIG. 7 is a block diagram of a controller that may be used in connection with any of the devices shown in FIGS. 1-3 and/or sensors shown in FIGS. 4, 5, 6, and 9;

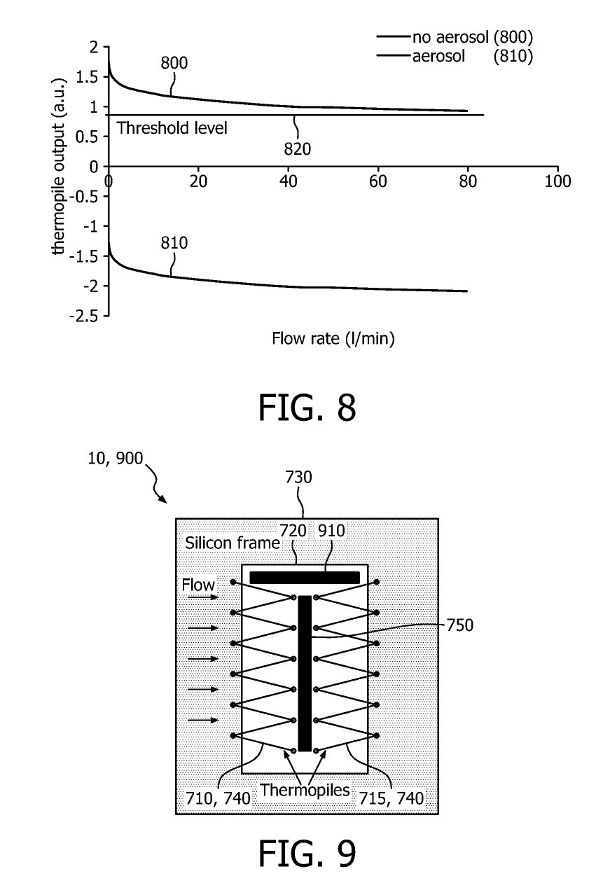

FIG. 8 is a graph of the thermopile output of the flow sensor in FIG. 6 versus flow rate past the thermal flow sensor according to an embodiment of the present invention;

FIG. 9 is a front view of a thermal flow sensor that may be used in connection with any of the devices shown in FIGS. 1-3 according to various embodiments of the present invention; and

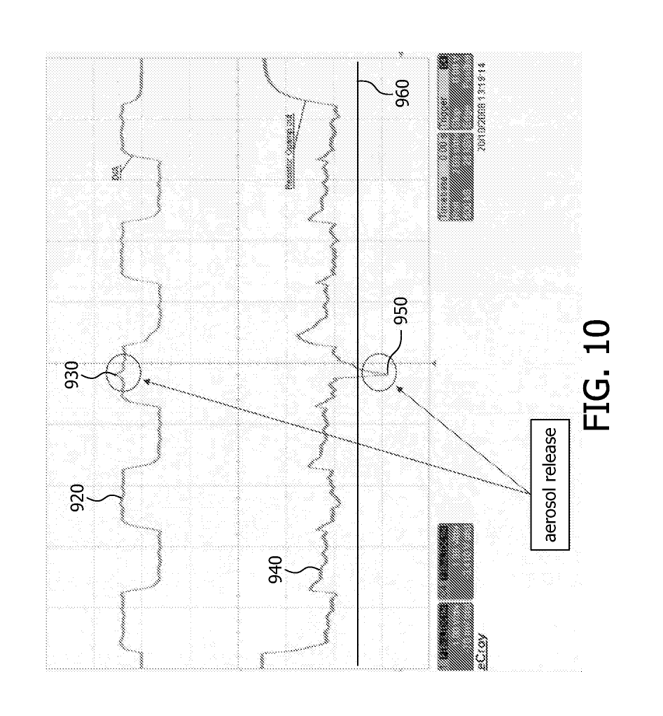

FIG. 10 is a graph of the temperature sensor output and flow sensor output of the flow sensor in FIG. 9 over time as a patient uses the device according to an embodiment of the present invention.

According to various embodiments of the present invention, an aerosol delivery system/device (e.g., an MDI 100 or a nebulizer 200, 300 (see FIGS. 1-3)) includes a sensor 10 that senses aerosol within the delivery system (e.g., sensors 400, 500, 700, 900 (see FIGS. 4-6 and 9)) and/or fluid flow through the aerosol delivery system (e.g., sensors 700, 900). The aerosol delivery system 100, 200, 300 also includes a controller 600 operatively connected to the sensor 10.

FIGS. 1-3 illustrate various aerosol delivery systems according to alternative embodiments of the present invention.

For example, as illustrated in FIG. 1, an aerosol delivery system according to an embodiment of the present invention comprises an MDI 100. The general features of this MDI 100 are described in U.S. Patent Application Publication No. 2004/0231665 A1, the entire contents of which are hereby incorporated herein by reference. The MDI 100 includes an aerosol generator 110 that is constructed and arranged to connect to a canister 120 of pressurized medicament. The aerosol generator 110 is constructed and arranged to generate aerosol by selectively releasing from the canister 120 a bolus of aerosolized medicament into a spacer 130 of the MDI 100 when a user pushes the canister 120 downwardly toward the aerosol generator 110. The MDI 100 also includes an aerosol output opening 140 disposed on an opposite side of the spacer 130 from the aerosol generator 110.

In the illustrated embodiment, the MDI 100 includes a spacer 130. However, the spacer 130 may be omitted without deviating from the scope of the present invention.

In the illustrated embodiment, the aerosol output opening 140 comprises a face mask 150. However, any other suitable aerosol output openings 140 may be used in place of a face mask 150 (e.g., a straw-like mouth piece, a ventilator tube, etc.) without deviating from the scope of the present invention.

A fluid pathway 160 extends from the aerosol generator 110 to the aerosol output opening 140. The sensor 10 is mounted to the MDI 100 at a location in which the sensor 10 can sense a temperature of the pathway 160. For example, the sensor 10 may be disposed within the pathway 160 (e.g., between the aerosol generator and the spacer 130, inside the spacer 130, between the spacer 130 and the aerosol output opening 140). The sensor 10 may alternatively be disposed in or on a wall that defines the pathway 160 (e.g., in a wall of the spacer 130 or aerosol generator 110). The sensor 10 may alternatively be disposed in any location that enables the sensor 10 to quickly follow temperature fluctuations in the pathway 160.

As illustrated in FIG. 2, an aerosol delivery system according to an embodiment of the present invention comprises a jet nebulizer 200. The general features of this nebulizer 200 are described in U.S. Patent Application Publication No. 2005/0087189 A1, the entire contents of which are hereby incorporated herein by reference. The nebulizer 200 comprises a jet-based aerosol generator 210 that relies on a stream of pressurized gas to aerosolize fluid 215 held in a container 220. A series of passageways 230 extend from the aerosol generator 210 to an aerosol output opening 240 and define a fluid pathway 260. In the illustrated embodiment, the aerosol output opening comprises a mouthpiece 250.

As shown in FIG. 2, the sensor 10 is mounted to the nebulizer 200 at a location in which the sensor 10 can sense a temperature of the pathway 260. For example, the sensor 10 may be disposed within the pathway 260 (e.g., between the aerosol generator 210 and the aerosol output opening 240). The sensor 10 may alternatively be disposed in or on a wall that defines the pathway 260. The sensor 10 may alternatively be disposed in any location that enables the sensor 10 to quickly follow temperature fluctuations in the pathway 260.

As illustrated in FIG. 3, an aerosol delivery system according to an embodiment of the present invention comprises an ultrasonic nebulizer 300. The general features of this nebulizer 300 are described in U.S. Patent Application Publication No. 2007/0277816 A1, the entire contents of which are hereby incorporated herein by reference. The nebulizer 300 is similar to the nebulizer 200, except that the aerosol generator 310 of the nebulizer 300 comprises an ultrasonic transducer 310 instead of a jet nebulizer to aerosolize fluid 315 in a container 320. Specifically, the transducer 310 propagates ultrasonic energy into the fluid 315, which causes the fluid 315 to aerosolize at the surface of the fluid 315. A series of passageways 330 extend from the aerosol generator 310 to an aerosol output opening 340 and define a fluid pathway 360. As explained above with respect to the nebulizer 200, the sensor 10 may be placed in any suitable location (e.g., in the pathway 360, in or on a wall that defines the pathway 360, in location that enables the sensor 10 to quickly follow temperature fluctuations in the pathway 360).

According to an alternative embodiment, the aerosol generator 310 is replaced with an aerosol generator that uses an ultrasonic, vibrating mesh plate to aerosolize fluid by forcing small droplets of the fluid through the mesh as the mesh vibrates.

FIGS. 4-6 illustrate three different temperature sensors 400, 500, 700 which may be used as the sensor 10 of the aerosol delivery devices 100, 200, 300.

FIG. 4 illustrates a temperature sensor 400. The sensor 400 comprises a temperature sensitive resistor 410 whose resistance varies with temperature. The resistor 410 is disposed on a membrane 420 that is suspended across an opening in a silicon frame 430 to create a base for the resistor 410. Thus, the resistor 410 is disposed on the base (e.g., attached to the base, integrally constructed with the base, formed in the base, abutting the base, etc.). The membrane 420 has a low thermal capacitance (e.g., lower than the silicon frame 430) such that the membrane 420 and resistor 410 will quickly follow temperature changes in the pathway 160, 260, 360.

FIG. 5 illustrates a temperature sensor 500 according to an alternative embodiment of the present invention. The sensor 500 uses a thermocouple 540 or multiple thermocouples in series (also known as a thermopile 510) instead of a resistor 410 to sense temperature. Like the sensor 400, the sensor 500 includes a base that comprises a membrane 520 that is suspended across an opening in a silicon frame 530. Each thermocouple 540 includes a reference junction 540a and a sensing junction 540b. The reference junction 540a is disposed on and senses a temperature of the silicon frame 530. The sensing junction 540b is disposed on and senses a temperature of the membrane 520. Because the membrane 520 has a lower thermal capacitance than the frame 530, the membrane 520 will follow temperature changes in the fluid passing the sensor 500 in the pathway 160, 260, 360 much more quickly than the silicon frame 530. Consequently, temperature changes in the pathway 160, 260, 360 will result in temperature differentials between the silicon frame 530 and membrane 520, for which the thermocouples 540 will generate a proportional voltage difference over the thermocouples 540.

In the illustrated embodiments, the reference junctions 540a are disposed in a location that may follow (albeit less quickly) the temperature of the pathway 160, 260, 360. According to an alternative embodiment, the reference junctions 540a may be spaced from the pathway 160, 260, 360 sufficiently far that the temperature at the junctions 540a is less affected by the temperature in the pathway 160, 260, 360. Such spacing may provide a more accurate, higher signal-to-noise-ratio signal. However, such spacing may complicate production and increase costs of the sensor 500, which is otherwise preferably a stand alone, integrated unit.

FIGS. 4 and 5 illustrate two example temperature sensors 400, 500 according to various embodiments of the present invention. However, any suitable alternative temperature sensor may be used in place of these sensors 400, 500 as the sensor 10 without deviating from the scope of the present invention. For example, the temperature sensor 10 may comprise temperature-sensitive transistor(s) or an infrared temperature sensor. The temperature sensor 10 may be a PTAT circuit that is located on the membrane, and provides a signal that is proportional to absolute temperature.

As shown in FIG. 7, the controller 600 comprises a processor 610, visual display 620, an audio output device 630, a memory 640, a user input device 650, and a haptic output device 660. However, according to various embodiments of the present invention, the one or more of these controller 600 components (e.g., the display 620, the memory 640, the audio output device 630, the user input device 650, and/or haptic output device 660) may be omitted without deviating from the scope of the present invention.

Returning to the aerosol delivery systems 100, 200, 300 illustrated in FIGS. 1-3, the sensor 10 in the form of a temperature sensor 400, 500 operatively connects to a controller 600 as shown in FIG. 7 via suitable wires 615 (or other data transmission means such as wireless communication (e.g., rf transmission, inductive data transmission, etc.). The controller 600 connects to the sensor 400, 500 to receive from the sensor 400, 500 a temperature signal that correlates with the temperature of the pathway 160, 260, 360. For example, in the resistive sensor 400, temperature is correlated to a resistance of the resistor 410 of the sensor 400 such that the resistor's resistance is a temperature signal. The controller 600 can therefore determine the temperature at the resistor 410 by measuring the resistance across the resistor 410. In the thermocouple-based sensor 500, temperature (specifically a temperature differential between the reference junctions 540a and sensing junctions 540b) is correlated to a voltage generated by the thermocouples 540 of the thermopile(s) 510 such that the voltage is a temperature signal. The controller can therefore determine the temperature at the sensing junctions 540b (relative to the reference junctions 540a) by measuring the voltage across the thermocouples 540 and thermopile(s) 510.

As explained below, the controller 600 is constructed and arranged to use the sensed temperature/temperature signal (e.g., resistance of the resistor 410 of the sensor 400, voltage of the thermopile(s) 510 of the sensor 500) to detect the presence of aerosol in the fluid pathway 160, 260, 360.

As shown in FIG. 1, when the aerosol generator 110 releases a bolus of aerosolized medicament into the spacer 130, the pathway 160 temperature drops due to expansion of the released gases and the rapid evaporation of the volatile propellant components of the bolus. For example, the small droplets in the bolus of aerosol evaporate rapidly because of the large total surface of the droplets and the low boiling point of the propellant. Because evaporation is an endothermic process the aerosol withdraws energy from its environment thereby decreasing the temperature of the environment, specifically the gas in the pathway 160, 260, 360. Consequently, the temperature of the pathway 160, 260, 360 downstream of the aerosol generator 110, 210, 310 decreases as this aerosol passes by. The temperature sensor 10, 400, 500 senses this temperature drop.

As shown in FIG. 7, the processor 610 of the controller 600 operatively connects to the sensor 10, 400, 500 and monitors for temperature drops that result from a bolus release or the presence of aerosol in the pathway 160, 260, 360.

According to one embodiment, the controller 600 monitors the sensor 500 and determines that a bolus was released when the temperature signal exceeds a predetermined threshold (e.g., 1.0 a.u.). In the sensor 500, a magnitude of the sensor signal is proportional to a difference in temperature between the membrane 520 and the silicon frame 530. There will be a large temperature differential between the membrane 520 and silicon frame 530 when aerosol is present and cools down the membrane 520 faster than the silicon frame 530, due to the membrane's relatively lower thermal capacity.

The above-described sensor 500 may be ambient temperature insensitive because it senses a temperature differential between the membrane 520 and frame 530, rather than an absolute temperature. For example, regardless of whether the sensor 500 is used in a cold or hot ambient environment, as long as the sensor 500 is given enough time between any change in ambient temperature for the membrane 520 and frame 530 to equalize in temperature, the sensor 500 will sense no temperature differential in the absence of events in the pathway 160, 260, 360 that would cause a temperature differential (e.g., the presence of aerosol).

According to one or more embodiments of the temperature sensor, for example the sensor 400 or a mercury- or bimetallic-based thermometer, the controller 600 may establish a baseline temperature when the controller 600 is turned on shortly before the aerosol delivery system 100, 200, 300 is used. The controller 600 may store this sensed initial baseline temperature in its memory 640 and determine that aerosol is present when the subsequently sensed temperature deviates from (e.g., is colder than) the baseline temperature by more than a predetermined threshold.

According to an alternative embodiment, the controller 600 determines that a bolus was released when the controller detects a rapid temperature drop in the pathway 160. For example, the processor 610 may determine that a bolus was released if a time-based rate of temperature drop exceeds a predetermined threshold. For example, the processor 610 may determine that a bolus was released if a temperature signal drop of more than a predetermined threshold occurs within a predetermined timeframe. According to various embodiments, the temperature drop threshold (e.g., resistance change of the resistor 410, voltage change of the thermopile(s) 510) may correlate to a temperature drop of at least 1, 2, 3, 4, 5, 6, 7, 8, 9, 10, 11, 12, 13, 14, or 15 degrees Celsius. According to various of these embodiments, the predetermined timeframe for detecting the temperature drop threshold may be less than 0.1, 0.5, 1, 2, 3, 4, 5, 6, 7, 8, 9, or 10 seconds. However, depending on the type of pathway 160, type of aerosol generator, type of aerosol, expected fluid flow rate over the sensor 400, 500, and a variety of additional and/or alternative factors, these thresholds may be increased or reduced to facilitate more precise and/or accurate detection of the bolus release.

The processor 610 may be any suitable type of processor. For example, the processor 610 may comprise an integrated circuit. The processor 610 may be digital or analog. In the case of a digital processor 610, the processor 610 may include A/D converter(s) to convert an analog temperature signals into digital signals. The processor 610 may comprise a computer. The processor 610 may carry out its monitoring, calculating, and other functions via operation of a program on the computer (e.g., a computer executable medium having executable code that carries out the various functions of the processor 610). The processor 610 may comprise a combination of two or more discrete processors without deviating from the scope of the present invention.

The display 620 may be any type of suitable visual display (e.g., one or more LED indicators with permanent indicia on the controller 600 indicating the meaning of each LED, an LCD screen capable of displaying text and/or graphical indicia). The processor 610 connects to the display 620 to display various information. For example, the processor 610 may provide a visual indication via the display 620 each time a bolus is released.

As shown in FIG. 7, the processor 610 may additionally and/or alternatively cause the audio output device 630 to indicate to the user when a bolus is released. The audio output device 630 may be any suitable type of noise-generating device (e.g., speaker, buzzer, etc.). The audio indication may be a beep to let the user know that a bolus was released. The audio indication may alternatively comprise spoken words (e.g., “A dose of medication has been released.”).

As shown in FIG. 7, in addition to or in the alternative to visual and audible signals, the controller 600 may include a haptic indicator 660 (e.g., a vibrator that uses a motor and offset flywheel) to provide haptic feedback to the user (e.g., vibrating when a bolus is released; vibrating when a fault is detected, etc.). Thus, the controller 600 may provide a bolus release indicator that provides audio, visual, and/or haptic indication to the patient when a bolus is released.

The processor 610 may be used to help a user coordinate their use of the system 100 with the release of the bolus. For example, at a predetermined time after the processor 610 detects a bolus release, the processor 610 may provide a visual indication (via the display 620) and/or audio indication (via the audio output device 630) and/or haptic indication (via the haptic output device 660) that the patient should inhale through the aerosol output opening 140. The predetermined time may be any suitable time (e.g., 0 seconds, 1 second, 2 seconds). For example, at the predetermined time after determining a bolus was released, the processor 610 may cause the audio output device 630 to say to the user “Inhale through the mouthpiece now.”

The processor 610 may have an incremental counter function that counts the number of boluses released. The processor 610 may cause the display 620 to visually indicate the number of boluses released. The processor 610 may connect to a memory 640 and use the memory 640 to store information obtained via the processor 610 and sensor 10. For example, the memory 640 may be used to store the incremental number of boluses released. The processor 610 may also include a time/date clock and function that associates bolus releases with the time and date of the release. The processor 610 may store this logged time/date/release data in the memory 640. The processor 610 may cause the display 620 to display such information. For example, the processor 610 may cause the display 620 to indicate the time and/or date of the last bolus release. Such historical data may help patients keep track of use of the system 100 and know when they should next use the system 100. The processor 610 may itself keep track of when the patient should receive the next medication dose and provide the patient with a visual, audible, and/or haptic indication when it is time for the next dose.

As shown in FIG. 7, the controller 600 may include a user input device 650 connected to the processor 610. The user input device 650 may comprise any suitable device for enabling a user to provide information to the controller 600. For example, the user input device 650 may comprise one or more buttons like a keypad or keyboard. The user input device 650 may comprise a touch screen input device incorporated into the display 620. One of the buttons/switches of the user input device 650 may be an on/off switch for the controller 600.

The user input device 650 may be used to provide a variety of information to the controller 600. For example, the user input device 650 may have a counting reset button that a user presses whenever the user replaces a used medication canister 120 with a new canister 120. Upon receiving a reset signal via the input device 650, the processor 610 may reset the counter to 0 so as to restart counting of how many boluses of medication have been released from the canister 120.

The processor 610 may be constructed and arranged to indicate to the user when the canister 120 is nearly empty (e.g., providing an indication when the count exceeds a predetermined threshold) so that the user knows to either replace the canister 120 or make preparations to have a fresh canister available. The threshold (or some other data by which the controller 600 can calculate the appropriate threshold) may be entered into the controller 600 via the user input device 650 by the user based on the type of canister 120 being attached to the system 100. Alternatively, the controller 600 may determine such information via the canister 120 itself (e.g., an RFID on the canister).

According to an alternative embodiment of the present invention, the processor 610 may use information relating to the number of doses in a canister 120 to decrement a counter that is displayed on the display 620. Consequently, the counter would illustrate approximately how many doses remain in the canister 120.

The controller 600 may connect to an activation mechanism of the aerosol generator 110 such that the processor 610 can determine when the activation mechanism has been activated. For example, the controller may use a pressure switch that detects when the canister 120 is pushed to release a bolus. Upon receipt of such an activation signal, the processor 610 can then determine from the sensor 10 if a bolus has actually been released. If the activation mechanism has been triggered but no bolus is sensed, the processor 610 may provide a visual or audible signal to the user that a fault has occurred (e.g., the aerosol generator malfunctioned, the canister 120 is empty).

As shown in FIGS. 2 and 3, the controller 600 may serve similar functions in connection with the nebulizers 200, 300. For example, the processor 610 may use the temperature signal to detect the presence of aerosol in the pathway 260, 360 in the same or similar manner as explained above with respect to the detection of the release of a bolus in the system 100.

For example, when the aerosol generator 210, 310 starts aerosolizing fluid from the container 220, 230, evaporation of the aerosolized droplets will quickly reduce the temperature of the pathway 260, 360 where the aerosol is present. As explained above, the processor 610 can determine that aerosol is present in the pathway 260, 360 (and therefore that the aerosol generator 210, 310 is aerosolizing liquid) when a rapid temperature drop is detected (e.g., a temperature drop exceeding a predetermined temperature differential threshold over a predetermined time).

Conversely, a rapid temperature increase indicates that the aerosol generator 210, 310 has ceased aerosolization of the fluid in the container 220, 230. The processor 610 can detect the cessation of aerosolization by detecting this rapid temperature rise. For example, the processor 610 can determine that aerosol generation has ceased when a rapid temperature increase is detected (e.g., a temperature rise exceeding a predetermined temperature differential threshold over a predetermined time). The temperature differential and predetermined time used to detect the cessation of aerosolization (and the accompanying absence of aerosol in the pathway 260, 360) may be the same as or different than the thresholds used to detect the start of aerosolization.

Alternatively, the controller 600 may use any other suitable method for detecting the start and/or stop of aerosolization from the temperature signal (e.g., any method described above with respect to the MDI 100 such as detecting when the temperature deviates from a baseline temperature by more than a predetermined threshold).

The processor 610 may provide a visual indication (via the display 620), an audio indication (via the audio output device 630), and/or a haptic indication (via a haptic output device 660) when aerosol is present in the pathway 260, 360. The controller 600 may indicate to the user when the aerosol generator 210, 310 begins aerosolizing fluid in the container 220, 320 and/or stops aerosolizing fluid from the container 220, 320 (e.g., when the container 220, 320 has run dry). For example, the controller 600 may visually, audibly, and/or haptically direct the patient to inhale from the aerosol output opening 240, 340 when aerosol is detected in the pathway 260, 360.

Because a typical dose for a nebulizer requires the patient to continue to use the system 200, 300 until all medication/liquid has been aerosolized, the controller 600 may indicate to the user to continue to breath through the aerosol output opening 240, 340 until the processor 610 detects that the container 220, 320 has run dry by detecting that aerosol is no longer being generated by the aerosol generator 210, 310. The controller 600 may visually, audibly, and/or haptically indicate to the user to stop using the nebulizer 200, 300 once the run dry is detected. For example, the audio output device 630 may verbally instruct the patient that “Dose complete—You may now stop using the nebulizer.” The controller 600 may automatically turn off the aerosol generator 210, 310 when run dry is detected.

As used herein, the term “run dry” means that substantially all aerosolizable fluid in the container 220, 320 has been aerosolized such that continued operation of the aerosol generator 21, 310 aerosolizes an insignificant amount of additional fluid (e.g., such that the aerosol output is less than 20%, 15%, and/or 10% of the normal output when sufficient fluid is in the container 220, 320). Thus, a container 220, 320 can “run dry” even though some fluid remains in the container 220, 320.

Some nebulizers coordinate nebulization with the patient's breathing cycle, e.g., to only aerosolize medication when the patient is inhaling or at desired portions of the patient's inhalation. In such nebulizers, the processor 610 may determine that the container 220, 320 has only run dry when the aerosol generator 210, 310 is operating but aerosol is still not detected in the pathway 260, 360.

As with the MDI 100, the controller 600 may be used in connection with a nebulizer 200, 300 to record usage data. For example, the processor 610 may record in the memory 640 the time, date, and/or duration of each use of the nebulizer 200, 300. The processor 610 may display logged data on the display 620 (e.g., time and/or date of last use, scheduled time for next use, etc.). The memory 640 may be accessible by the user and/or medical provider to facilitate analysis of the logged data.

In the embodiment shown in FIG. 1, the controller 600 is mounted to the remainder of the MDI 100. In the embodiments shown in FIGS. 2 and 3, the controller is separate from the systems 200, 300, but tethered to the systems via the connecting wire 615. According to alternative embodiments of the present invention, the controller 600 may have any other suitable physical relationship to the remainder of the system 100, 200, 300 without deviating from the scope of the present invention (e.g., be incorporated into the housing of any system or be separate from the remainder of the system).

FIG. 6 illustrates a thermal flow sensor 700, which may be used as the sensor 10 in connection with various embodiments of the present invention, including the aerosol delivery systems 100, 200, 300. The thermal flow sensor 700 comprises an upstream temperature sensor 710, a downstream temperature sensor 715, a base that includes a membrane 720 suspended across an opening in a silicon frame 730, and a heater 750 centrally disposed on the membrane 720.

According to one or more embodiments, the sensor 400, 500, 700 (including the frame 430, 530, 730, the membrane 420, 520, 720, and the various electrical components 410, 510, 710, 715, 750) is manufactured using known chip/semiconductor manufacturing techniques. The sensor 400, 500, 700 may be manufactured using the method disclosed in the attached patent application titled “THERMAL FLOW SENSOR INTEGRATED CIRCUIT WITH LOW RESPONSE TIME AND HIGH SENSITIVITY,” the entire contents of which are hereby incorporated by reference.

The base defines upstream and downstream directions, the downstream direction being indicated in FIG. 6 by the flow direction arrows. According to various embodiments, the sensor 700 is positioned relative to the pathway 160, 260, 360 such that the downstream direction of the sensor 700/base is aligned with the direction of fluid flow as fluid flows from the aerosol generator 110, 210, 310 toward the aerosol output opening 140, 240, 340. In other words, the downstream direction of the base is directed along the fluid pathway 160, 260, 360 toward the aerosol output opening 140, 240, 340 such that sensed flow in the downstream direction of the sensor 700 indicates fluid flow in the pathway 160, 260, 360 toward the aerosol output opening 140, 240, 340 (i.e., indicating inhalation by a patient), and, conversely, sensed flow in the upstream direction of the sensor 700 indicated fluid flow in the pathway 160, 260, 360 toward the aerosol generator 110, 210, 310 (i.e., indicating exhalation by the patient in a system in which the sensor 700 is positioned such that exhalation gases pass the sensor 700).

The heater 750 connects to the controller 600 so as to receive current from the controller 600, which heats the heater 750. The heater 750 may be any suitable heater, e.g., a resistor. The heater 750 heats up the membrane 720 thereby creating a temperature profile which is maximal in the center at the location of the heater 750 and minimal at the silicon frame 730 which acts as a heat sink.

During operation of the sensor 730, the controller 600 may provide a constant current to the heater 750. However, according to alternative embodiments, the controller 600 may vary the current without deviating from the scope of the present invention.

The illustrated temperature sensors 710, 715 comprise thermopiles 710, 715 that each comprise a plurality of thermocouples 540 that each include a reference junction 740a and a sensing junction 740b. The reference junctions 740a are disposed on and sense a temperature of the silicon frame 730. The sensing junctions 740b of the upstream temperature sensor 710 are disposed on and sense an upstream temperature of the membrane 720 at a location upstream from the heater 750. The thermopile 710 therefore generates an upstream temperature signal in the form of a voltage that is proportional to a temperature differential between the silicon frame 730 at the reference junctions 740a of the thermopile 710 and the sensing junctions 740b of the thermopile 710 upstream from the heater 750.

The sensing junctions 740b of the downstream temperature sensor 715 are disposed on and sense a downstream temperature of the membrane 720 at a location downstream from the heater 750. The thermopile 715 therefore generates a downstream temperature signal in the form of a voltage that is proportional to a temperature differential between the silicon frame 730 at the reference junctions 740a of the thermopile 715 and the sensing junctions 740b of the thermopile 715 downstream from the heater 750.

Because the membrane 720 has a lower thermal capacitance than the frame 730, the membrane 720 will follow temperature changes in the fluid passing the sensor 700 in the pathway 160, 260, 360 much more quickly than the silicon frame 730. Consequently, temperature changes in the pathway 160, 260, 360 will result in temperature differentials between the silicon frame 730 and membrane 720, for which the thermocouples 740 will generate a proportional voltage difference.

According to one or more embodiments, the membrane 420, 520, 720 comprises a substrate that quickly follows temperature changes in the pathway 160, 260, 360 (e.g., a material with a low thermal capacity). For example, the membrane 420, 520, 720 may comprise a relatively thin layer of material that has a low thermal capacity such that it quickly responds to temperature changes in the surrounding environment. According to various embodiments, the membrane 420 comprises silicon, silicon nitride, silicon oxide, polyimide, parylene, and/or glass. Such characteristics may improve the ratio of flow-dependent temperature differences to dissipated power in the heater 750.

In the illustrated embodiment, the frame 430, 530, 730 comprises silicon. However, the frame 430, 530, 730 may alternatively comprise any other suitable material. According to one or more embodiments, the frame 430, 530, 730 comprises a material that follows temperature changes in the pathway 160, 260, 360 more slowly than the membrane 420, 520, 720 (e.g., a thicker material and/or material with a higher thermal capacity than the membrane 420, 520, 720), if at all.

In various embodiments, temperature variations between the upstream and downstream sides of the silicon frame 730 are small relative to temperature differences between the upstream and downstream sides of the membrane 720 due to the high relative thermal diffusivity of the silicon frame 730. As a result, the temperature difference between the upstream and downstream sides of the silicon frame 730 (i.e., where the reference junctions 704a are disposed) is much smaller than the temperature variations in the membrane 720 (i.e., where the sensing junctions 740b are disposed) and can therefore be neglected according to one or more embodiments of the present invention.

As shown in FIG. 6, the temperature sensors 710, 715 are disposed thermally symmetrically upstream and downstream, respectively, from the heater 750. In an embodiment where the heater 750 is centrally disposed on the membrane 720 and the upstream and downstream heat capacity and diffusivity of the of the membrane 720 is symmetrical relative to the heater 750, an upstream distance between the upstream temperature sensor 710 and heater 750 may be substantially equal to a downstream distance between the downstream temperature sensor 715 and the heater 750.

As a result of such symmetrical placement of the sensors 710, 715, in the absence of fluid flow in the upstream/downstream direction past the sensor 700, while the heater 750 is on, the upstream and downstream temperatures (as well as the upstream and downstream temperature signals) will be substantially equal to each other (e.g., within 10, 5, 4, 3, 2, or 1 degrees Celsius of each other). When fluid flows downstream past the sensor 700 while the heater 750 is on, the downstream temperature will rise relative to the upstream temperature as the flow pushes/carries heat from the heater 750 downstream away from the upstream sensor 710 and toward the downstream sensor 715. Conversely, when fluid flows upstream past the sensor 700 while the heater 750 is on, the downstream temperature will fall relative to the upstream temperature as the flow pushes heat upstream away from the downstream sensor 715 and toward the upstream sensor 710. It should be noted, however, that fluid flow in either direction may cause the absolute upstream and downstream temperatures to drop as the flow cools the pathway 160, 260, 360 and sensor 700 more than the heater 750 heats the membrane 720.

A magnitude of the temperature differential between the upstream and downstream temperatures will be proportional to a magnitude of the fluid flow rate because a faster fluid flow rate will push/carry more heat in the direction of flow. In the illustrated embodiment, a flow sensor temperature differential is defined in terms of a voltage differential in the thermopiles 710, 715, which is correlated to the actual upstream and downstream temperatures. The sign of the flow sensor temperature differential indicates a direction of flow past the sensor 700 in an embodiment where the sensors 710, 715 are thermally symmetrically disposed relative to the heater 750. For example, if the polarity of the sensors 710, 715 is set up so that they register positive polarity voltage when the sensing junctions 740b are colder than the reference junctions 740a, the flow sensor temperature differential (e.g., a voltage differential defined as the upstream sensor 710 voltage signal minus the downstream sensor 715 voltage signal) will have a positive polarity when flow is downstream, and a negative polarity when flow is upstream. An absolute magnitude of the differential (e.g., a magnitude of the voltage) is proportional (typically, but not necessarily, non-linearly) to the absolute flow rate past the sensor 700.

Thermally symmetrical placement of the upstream and downstream sensors 710, 715 relative to the heater 750 may result in (a) an offset free flow rate determination (no flow gives zero signal), (b) the ability to determine flow direction from the sign of the differential signal, (d) upstream and downstream flow rates being identically correlated to the absolute value of the differential signal. Due to the symmetry of the sensors 710, 715, the differential signal (e.g., the flow rate signal) may also be insensitive for variations in ambient temperature. This is because both thermopile 710, 715 signals change with the same absolute amount, which cancels when subtracting or dividing the two signals.

Although the sensors 710, 715 are symmetrically disposed upstream and downstream, respectively, from the heater 750 in the illustrated sensor 700, the upstream sensor 710 may be alternatively disposed according to alternative embodiments of the present invention. For example, if only downstream flow is desired to be measured, the upstream sensor 710 may be disposed in a section of the pathway 160, 260, 360 that is far from and generally unaffected by the heater 750. However, for the reasons explained herein, according to one or more embodiments, symmetrical placement of the sensors 710, 715 tends to improve calibration, accuracy, and precision, among other things.

Although the illustrated temperature sensors 710, 715 comprise thermopiles, the temperature sensors may alternatively comprise any other suitable type of temperature sensors without deviating from the scope of the present invention.

Although a particular flow sensor 700 is described herein, a variety of alternative flow sensors could be used in conjunction with various embodiments of the present invention without deviating from the scope of the present invention.

The controller 600 may be constructed and arranged to use the thermal flow sensor 700 in various ways. As shown in FIG. 7, the controller 600 is connected, via the wires 615, to the sensor 10, 700. As explained above, the controller 600 delivers current to the heater 750 via these wires 615. The controller 600 also connects to the sensors 710, 715 via the wires 615 to receive from the sensors 710, 715 upstream and downstream temperature signals, respectively, that correlate to the upstream and downstream temperatures, respectively. The controller 600 compares the upstream and downstream temperature signals to detect fluid flow within the pathway 160, 260, 360 by comparing the upstream and downstream temperature signals.

The controller 600 is constructed and arranged to determine the presence and direction of fluid flow within the pathway 160, 260, 360 by comparing the upstream and downstream temperatures/signals. For example, if the controller 600 determines that the upstream and downstream temperatures are approximately equal, the controller 600 determines that there is no fluid flow through the pathway 160, 260, 360. If the controller 600 determines that the downstream temperature has risen relative to the upstream temperature (or is higher than the upstream temperature in various thermally symmetrical embodiments), the controller 600 (or the processor 610 thereof) determines that fluid is flowing downstream toward the aerosol output opening 140, 240, 340. Conversely, if the controller 600 determines that the downstream temperature has fallen relative to the upstream temperature (or is lower than the upstream temperature in the case of various thermally symmetrical embodiments), the controller 600 (or the processor 610 thereof) determines that fluid is flowing upstream toward the aerosol generator 110, 210, 310.

The controller 600 may compare the upstream and downstream temperatures/signals in any suitable manner. For example, the controller 600 may subtract the upstream temperature from the downstream temperature and use the sign of the result to determine the direction of flow, with a result of zero indicating no fluid flow. Alternatively, the controller 600 may compare the upstream and downstream temperatures/signals by dividing one by the other and determining the flow direction by whether the quotient is greater than or less than one, with a quotient of one indicating that there is no flow.

The controller 600 may also use the sensor 700 to determine a fluid flow rate past the sensor 700. The determined fluid flow rate need not be in absolute terms (e.g., meters/second or liters/second). Rather, the fluid flow rate may be determined and expressed in terms of a variable that is correlated to the fluid flow rate. For example, in an embodiment in which the controller 600 subtracts the upstream temperature signal from the thermopile 710 (in terms of volts) from the downstream temperature signal from the thermopile 715 (in terms of volts), the resulting fluid flow rate may be expressed in volts (or any other suitable absolute or relative scale based on the type of temperature sensors used). The controller 600 may determine an actual volumetric flow rate in the pathway 160, 260, 360 or actual linear flow rate of fluid past the sensor 700 via a predetermined conversion algorithm that associates various temperature differential signals (e.g., in terms of volts) with actual flow rates (e.g., meters/second, liters/second, etc.). The algorithm may be mathematically calculated or may alternatively be generated empirically through controlled testing that determines the temperature differential signal at known flow rates.

The controller 600 may also use one or both of the temperature sensors 710, 715 of the sensor 700 as a temperature sensor similar to the above-discussed thermopile 510 of the sensor 500. For example, if both sensors 710, 715 are used, their signals may be added together to create a signal that varies with temperature. The sensor 700 can therefore be used in a manner similar to the sensor 500 to detect the presence of aerosol in the pathway 160, 260, 360.

During operation of the flow sensor 700 the heater 750 heats up the membrane 720, which is cooled by the airflow past the sensor 700. As illustrated in FIG. 8, the minimum temperature of the membrane 720 is reached at the maximum flow rate and vice versa. In FIG. 8, the y-axis (“thermopile output (a.u.)”) represents the cumulative temperature signal from both sensors 710, 715 according to one embodiment of the sensor. The x-axis represents flow rate. As shown in FIG. 8, the cumulative temperature signal/cumulative temperature is inversely proportional to flow rate. The cumulative signal is positive because the heater 750 heats the sensing junctions 740a relative to the reference junctions 740b.

As shown in FIG. 8, the cumulative temperature signal also varies with the presence of aerosol. The temperature v. flow rate curve 800 (the curve at the top of FIG. 8) is an example curve when no aerosol is present in the pathway in which the sensor 700 is positioned. The temperature v. flow rate curve 810 (the curve at the bottom of FIG. 8) is an example curve when aerosol is present in the pathway being sensed by the sensor 700. The temperature variation of the membrane 720 is determined by the amount of heat that is dissipated in the heater 750, e.g. a small amount of power gives small changes in temperature with varying flow. When aerosol is present, the temperature of the heated membrane 720 will cool down. For small heater 750 dissipation levels, the presence of aerosol will cool the membrane 720 below the temperature at the maximum flow rate in the absence of aerosol. In other words, all other variables being constant, the cumulative temperature at zero flow rate with aerosol present will be lower than the cumulative temperature at maximum flow rate in the absence of aerosol. A threshold level 820 is set just below the minimum temperature at the maximum flow rate in the absence of aerosol. The passing by of the aerosol is detected when the temperature drops below this threshold level 820. In the illustrated sensor 700, the cumulative temperature signal will be negative when the membrane 720 is colder than the silicon frame 730. In the illustrated sensor 700, the cumulative temperature signal will be positive in the absence of aerosol because the heater 750 heats sensing junctions 740b of the sensors 710, 715 near the heater 750 on the membrane 720 relative to the reference junctions 740a farther from the heater 750 on the silicon frame 730.

The heater 750 heat output can be optimized to balance competing variables. As explained above, reducing the heater 750 output makes it easier to differentiate between fast flow rates in the absence of aerosol and slow flow rates in the presence of aerosol. On the other hand, the heater 750 output can also be optimized to maximize the difference between the upstream and downstream temperatures during expected flow rates in order to optimize the signal-to-noise ratio of the sensor's ability to detect and quantify flow rates.

According to an alternative embodiment, the controller 600 utilizes an adaptive temperature threshold 820 to more accurately detect the presence of aerosol. As shown via the curve 800 in FIG. 8, a relation between cumulative temperature signal of the membrane 720 (relative to the silicon frame) and flow rate is known when aerosol is not present. Because the controller 600 can use the sensor 700 to calculate the flow rate as explained above by comparing the upstream and downstream temperature signals, the controller 600 can use the known flow rate along with the known cumulative-temperature-signal-to-flow-rate (in the absence of aerosol) relationship to determine what the cumulative temperature signal would be in the absence of aerosol. The controller 600 can therefore set the adaptive aerosol-detecting temperature signal to be slightly below the expected signal at the known flow rate in the absence of aerosol. The controller 600 determines that aerosol is present if the sensed cumulative temperature signal falls below the instantaneous adaptive threshold (in an embodiment where the temperature signal rises and falls with membrane 720 temperature). Thus, the adaptive threshold 820 will reduce with sensed flow rate. According to one or more embodiments that use an adaptive threshold 820, the difference between the actual membrane 720 temperature and the threshold level 820 can be small and thus smaller temperature drops (and therefore smaller amounts of aerosol) can be detected. Also, according to one or more embodiments that use an adaptive threshold 820, the adaptive threshold level 820 facilitates the use of a higher heater 750 heat output, which may increase the signal-to-noise ratio of the sensor's ability to sense gas flow. According to one or more embodiments that use an adaptive threshold 820, no maximum flow rate needs to be defined to determine the minimum temperature to set the threshold level 820.

FIG. 9 illustrates a thermal flow sensor 900 according to an alternative embodiment of the present invention. The sensor 900 may be used in place of any of the sensors 400, 500, 700 described herein without deviating from the scope of the present invention. The sensor 900 is identical to the sensor 700, except that a discrete temperature sensor 910 is added and mounted to the membrane 720. In the illustrated embodiment, the sensor 900 is a resistive temperature sensor like the above-described resistor 410 of the sensor 400. Alternatively, a sensor like the sensor 900 could be manufactured by actually using both the sensor 400 and the sensor 700.

The controller 600 connects to the resistive temperature sensor 910 in a similar manner that the controller 600 connects to the above-discussed resistor 410 of the sensor 400. The controller connects to the heater 750 and sensors 710, 715 in a similar manner as discussed above with respect to the sensor 700. The use of such a resistive temperature sensor 910 may enable the sensor to measure absolute temperature (as opposed to relative temperature using sensors such as thermocouples).

FIG. 10 illustrates the experimental results of the use of the controller 600 to sense the temperature and flow in a pathway using the sensor 900. The x-axis represents time. The top line 920 indicates the response of the flow sensor 900 to a user's breathing pattern (about five full breaths are shown). The y-axis of the line 920 is correlated to a temperature differential between the upstream and downstream temperature sensors 710, 715 (e.g., in terms of actual temperature (e.g., degrees Celsius), temperature signal differential (e.g., volts if the sensor 710, 715 are thermopiles, ohms for the resistive upstream and downstream temperature sensors)). In the line 920, the lower flat portions represent one of inhaling and exhaling, while the upper flat portions represent the other of inhaling and exhaling (depending on whether the sensor 900 is set up to subtract the upstream temperature from the downstream temperature or vice versa). When the aerosol is released a small spike 930 is observed in the flow sensor 900 signal showing the flow sensor 900 is hardly affected by the aerosol.

In FIG. 10, the lower line 940 is correlated to the temperature sensed by the resistor 910 (which may also be referred to as a thermistor), such that the y-axis of the line 940 is correlated to pathway temperature (e.g., in terms of resistance in ohms, in terms of actual temperature). The noisy pattern of the line 940 is caused by the temperature fluctuations of the heater 750 caused by changes in the flow. When the aerosol is released, the resistance of the resistor 910 drops to a level 950 far below the minimum level if no aerosol is present. As explained above with respect to the sensor 700, the controller 600 may utilize a preset or adaptive temperature threshold 960, and determine that aerosol is present when the line 940/temperature signal crosses the threshold 960.

According to an alternative embodiment, the sensor 700 is used and the resistance of the heater 750, itself, rather than a discrete resistor 910, is used to sense temperature in the same manner as described above with respect to the sensor 900.

The thermal flow sensors 700, 900 may be used in connection with the aerosol delivery devices 100, 200, 300 to provide additional or alternative functionality to these devices.

For example, during use of the MDI 100, a user should properly time the release of a bolus relative to inhalation of the bolus. According to different intended uses, it may be desired for the patient to inhale immediately upon (or a predetermined amount of time after) the bolus is released, or release the bolus during inhalation. As explained above, the controller 600 can use the sensors 700, 900 to detect the release of a bolus of aerosolized medication. Moreover, because the controller 600 can use the sensors 700, 900 to detect the presence, direction, and/or magnitude of flow in the pathway 160, the controller 600 can determine when the user is inhaling through the aerosol output opening 140. The controller 600 is therefore able to monitor patient compliance with the desired release/inhalation timing and/or provide instructions to the patient to help the patient better time the release and inhalation.

With respect to monitoring, the controller 600 may record in the memory 640 the timed relationship between each bolus release and each inhalation (e.g., relative start time, stop time, duration). This stored data can then be accessed by the user or a medical professional to assess the patient's compliance with the desired use of the MDI 100.

The controller 600 may compare the sensed relationship between release/inhalation to a predetermined desired relationship, and provide an indication (e.g., visually via the display 620, audibly via the audio output device 630, and/or haptically via the haptic output device 660) as to whether the patient properly timed the release and inhalation. If the patient's timing was not proper, the controller 600 may provide an indication as to how the patient can better comply with the desired timing in the future (e.g., a visual or audible indication such as “Next time, please inhale sooner (or later) relative to releasing the aerosol”).

The controller 600 may additionally and/or alternatively provide a real-time indication to the patient regarding when to release the bolus and/or inhale. For example, if the bolus should be released midway (or some other desired point) through a patient's inhalation, the controller 600 may provide a visual, audible, or haptic instruction to activate the aerosol generator 110 when the controller 600 detects, via the flow sensor 700, 900, that the patient is midway through an inhalation. Alternatively, in embodiments in which the controller 600 is connected to the aerosol generator 110, 210, 310 in such a manner as to permit the controller 600 to turn the aerosol generator 110, 210, 310 on or off, the controller 600 may itself turn on the aerosol generator 110, 210, 310 when the controller 600 determines that it is appropriate relative to the sensed breathing pattern of the patient.

Alternatively, if it is desired for the patient to inhale a predetermined time after releasing the bolus, the controller 600 may provide an appropriately timed visual, audible, or haptic instruction to inhale.

In connection with the nebulizer 200, 300, the controller 600 may use the flow sensors 700, 900 in a similar manner as described above with respect to the MID 100. For example, the controller 600 may monitor and record in the memory 640 the time, duration, and relative timing of aerosolization by the aerosol generator 210, 310 and patient inhalation through the aerosol output opening 240, 340. This data may subsequently be used by the user, a medical professional, or other suitable person or machine to assess the patient's compliance with the desired treatment regime. The data may warrant instructing the patient to use the device 200, 300 differently, and/or warrant adjustments to how the device 200, 300 operates (e.g., adjusting the device's own operation by adjusting, for example, the time and timing of each aerosol release to better match the patient's breathing pattern).

As is known in the art, it is often desirable to coordinate the patient's breathing pattern to the aerosolization by the nebulizer 200, 300. For example, various nebulizers are designed to aerosolize medication when the patient is inhaling, but not when the patient is exhaling, so as to reduce waste of the medication, among other reasons. The controller 600 may use the flow sensor 700, 900 to detect inhalation and exhalation so as to time the activation of the aerosol generator 210, 310 accordingly. In such embodiments, the controller 600 is operatively connected to the aerosol generator 210, 310 so as to enable the controller to start and stop the aerosol generator 210, 310.

Although example aerosol delivery devices 100, 200, 300 with example aerosol generators 110, 210, 310 are described above, alternative types of aerosol delivery devices and aerosol generators may be substituted for these example devices 100, 200, 300 and/or generators 110, 210, 310 without deviating from the scope of the present invention.

In the illustrated embodiments, the sensor 10 is disposed at an example location in the aerosol delivery devices 100, 200, 300. However, the sensor 10 may be disposed in an alternative location without deviating from the scope of the present invention. For example, the sensor 10 may be repositioned so as to improve the sensor's ability to detect inhalation, exhalation, and/or aerosol. The position of the sensor 10 may be optimized to balance competing goals of sensing various conditions.

For example, in the device 100 illustrated in FIG. 1, placing the sensor 10 near the aerosol generator 110 may improve the sensor's ability to detect the presence of aerosol. However, in this position, the sensor 10 may be unable to detect patient exhalation because significant exhalation flow may not reach the sensor 10, particularly if an exhalation valve is disposed closer to the mouth piece 140. The sensor 10 could alternatively be disposed in a location that is well suited to detect such inhalation/exhalation flow (e.g., as shown in phantom in FIG. 1 as sensor 10a). However, such placement may involve a trade off with the sensitivity of the sensor 10 to detect aerosol because the placement of the sensor 10a is farther from the aerosol generator 110.

For the same reasons, the sensor 10 shown in FIG. 2 in connection with the device 200 could be repositioned as shown in phantom in FIG. 2 as sensor 10b. While such placement of the sensor 10b may improve the sensor's ability to detect patient exhalation and inhalation, such placement could reduce the sensor's sensitivity to the detection of aerosol because the sensor 10b is disposed farther from the aerosol generator 210.

Further still, in one or more embodiments, the sensor 10 may be used to detect flow, but not the presence of aerosol. In such embodiments, the sensor 10 may be disposed in a location that minimizes or eliminates its interaction with aerosol so as to minimize aerosol-based contamination of the sensor 10. For example, as shown in phantom via the sensor 10c in FIG. 2, the sensor 10c can be placed in the inhalation fluid pathway upstream from the aerosol generator 210 so as to sense inhalation without significant contamination from the aerosol generated downstream of the sensor 10c. Similarly, as shown in phantom via the sensor 10d in FIG. 2, the sensor 10d can be placed in the exhalation pathway to improve its ability to sense patient exhalation while limiting the sensor's exposure to contaminating aerosol.

Similar alternative locations for the sensor 10 in the device 300 in FIG. 3 may be utilized to improve sensitivity to the prioritized measurements (e.g., aerosol presence, inhalation, exhalation).

In the illustrated embodiments, the sensor positions 10b, 10c, 10d provide alternative locations for the sensor 10. However, according to further embodiments, the devices 100, 200, 300 may use multiple sensors 10, each sensor 10 focusing on a different measurement. For example, in the device 200, the device 200 may use the sensor 10 to detect aerosol, the sensor 10c to detect inhalation, and the sensor 10d to detect exhalation.

In the illustrated embodiments, the aerosol delivery devices 100, 200, 300 are designed to aerosolize a medicament and the aerosol output openings 140, 240, 340 are designed to facilitate delivery of the aerosolized medicament into the airway (e.g., throat, bronchial tubes, lungs) of a patient via the patient's mouth and/or ventilator tube. However, according to alternative embodiments of the present invention, aerosol delivery systems may have alternative functions (e.g., humidification, spreading of scented aerosol such as air fresheners) without deviating from the scope of the present invention. Additionally and/or alternatively, one or more embodiments of the present invention may be used in any system in which it would be desirable to sense the presence of aerosol at a given location and/or sense fluid flow (in terms of existence of flow, direction of flow, and/or magnitude of flow). For example, the flow sensors 700, 900 described herein could be used in a gas pipeline to sense flow. Thus, various embodiments of the present invention are not limited to use in the aerosol generation and/or delivery context.

The various temperature sensors described herein may sense temperatures in a pathway 160, 260, 360 either directly (e.g., sensor disposed in the pathway) or indirectly (e.g., sensor disposed in the wall of the pathway, such that the sensor senses a temperature in the pathway indirectly by sensing a temperature in the wall).

As used herein, sensing temperature does not require sensing an absolute temperature. Rather, sensing a temperature merely requires generating some type of signal or information that is correlated to temperature. For example, temperature measurements may be in terms of a temperature difference from a reference location (e.g., via the reference and sensing junctions of a thermocouple). Temperature measurements need not be converted into standard temperature units (e.g., Fahrenheit, Celsius, Kelvin). Rather, temperature measurements can merely be correlated (e.g., proportional, inversely proportion) to temperature, such that temperature measurements may be made in terms, for example, of ohms/resistance for a resistive temperature sensor or volts for a thermocouple temperature sensor.

As used herein, the terms starting and stopping of aerosolization are not absolute. Rather starting and stopping of aerosolization may be detected when aerosolization is above or below a predetermined threshold. For example, it may be determined that aersolization has stopped when aersolization has reduced, relative to the aerosolization that occurs during normal operation of an aerosol generator, below a predetermined threshold (e.g., less than 20%, 15%, 10% of the normal aerosolization).

The pathway 160, 260, 360 may comprise the air space through which gas/air moves from the aerosol generator 110, 210, 310 to the aerosol output opening 140, 240, 340. Alternatively, the pathway 160, 260, 360 may also the surfaces that define the air space through which gas/air moves from the aerosol generator 110, 210, 310 to the aerosol output opening 140, 240, 340. The pathway 160, 260, 360 may also include the walls that define the surfaces of the air space.

The foregoing illustrated embodiments are provided to illustrate the structural and functional principles of the present invention and are not intended to be limiting. To the contrary, the principles of the present invention are intended to encompass any and all changes, alterations and/or substitutions within the spirit and scope of the following claims.

1. A thermal flow sensor comprising:
a base defining upstream and downstream directions;
a heater disposed on the base;
a first temperature sensor positioned so as to sense a first temperature at a first location; and
a downstream temperature sensor disposed on the base downstream of the heater so as to sense a downstream temperature of the base downstream from the heater,
wherein the temperature sensors and heater are located relative to each other such that fluid flow past the base in the downstream direction increases a temperature differential between the first temperature and the downstream temperature.

2. The sensor of claim 1, wherein a magnitude of the temperature differential is proportional to a magnitude of the flow rate of the fluid past the base.

3. The sensor of claim 1, wherein the first temperature sensor comprises an upstream temperature sensor that is disposed on the base upstream of the heater so as to sense an upstream temperature of the base upstream from the heater.

4. The sensor of claim 3, wherein:
the temperature differential comprises the downstream temperature minus the upstream temperature,
the temperature differential is positive when fluid is flowing in one of the upstream and downstream directions past the base, and
the temperature differential is negative when fluid is flowing in the other of the upstream and downstream directions past the base.

5. The sensor of claim 3, wherein an upstream distance between the upstream temperature sensor and heater is substantially equal to a downstream distance between the downstream temperature sensor and the heater.

6. The sensor of claim 3, wherein the upstream and downstream temperature sensors are positioned such that when the heater is turned on and there is no fluid flow over the base, the upstream and downstream temperatures are substantially identical.

7. The sensor of claim 6, wherein the temperature sensors and heater are located relative to each other such that fluid flow in a downstream direction increases the downstream temperature relative to the upstream temperature.

8. The sensor of claim 7, wherein the temperature sensors and heater are located relative to each other such that fluid flow past the base in an upstream direction increases the upstream temperature relative to the downstream temperature.

9. The sensor of claim 3, wherein:
the base comprises a frame and a membrane connected to the frame,
the frame has a higher thermal capacitance than the membrane,
the heater is disposed on the membrane,
the downstream temperature sensor is positioned to sense a temperature of the membrane downstream from the heater, and
the upstream temperature sensor is positioned to sense a temperature of the membrane upstream from the heater.

10. The sensor of claim 3, wherein:
the base comprises a silicon frame and a membrane connected to the silicon frame,
the silicon frame has a higher thermal capacitance than the membrane,
the heater is disposed on the membrane,
the downstream temperature sensor comprises a thermocouple having a reference junction disposed on the silicon frame and a sensing junction disposed on the membrane downstream from the heater, and
the upstream temperature sensor comprises a thermocouple having a reference junction disposed on the silicon frame and a sensing junction disposed on the membrane upstream from the heater.

11. The sensor of claim 3 in combination with an aerosol delivery system, the aerosol delivery system comprising:
an aerosol generator;
an aerosol output opening;
a fluid pathway extending to the aerosol output opening, the aerosol generator being positioned such that aerosol generated by the aerosol generator enters the fluid pathway, wherein the thermal flow sensor is in thermal communication with the pathway, wherein the downstream direction of the base is directed along the fluid pathway toward the aerosol output opening;
a controller connected to the upstream and downstream temperature sensors to receive from the sensors upstream and downstream temperature signals, respectively, that correlate to the upstream and downstream temperatures, respectively,
wherein the controller is constructed and arranged to detect fluid flow within the pathway by comparing the upstream and downstream temperature signals.

12. The combination of claim 11, wherein the controller is constructed and arranged to determine a direction of fluid flow within the pathway by comparing the upstream and downstream temperature signals.

13. The combination of claim 12, wherein:
the sensor is disposed downstream from where aerosol generated by the aerosol generator enters the pathway; and
the controller is constructed and arranged to use a temperature sensor signal from the sensor to detect the presence of aerosol in the fluid pathway.

14. The combination of claim 11, wherein:
the sensor is disposed upstream from where aerosol generated by the aerosol generator enters the pathway; and
the controller is constructed and arranged to detect inhalation flow within the pathway by comparing the upstream and downstream temperature signals.

15. The combination of claim 11, wherein:
the sensor is disposed downstream from where aerosol generated by the aerosol generator enters the pathway; and
the controller is constructed and arranged to use a temperature sensor signal from the sensor to detect the presence of aerosol in the fluid pathway.

16. The combination of claim 15, wherein the controller is constructed and arranged to determine that aerosol is present in the fluid pathway when the temperature sensor signal indicates a temperature below a predetermined temperature threshold.

17. The combination of claim 16, wherein the predetermined temperature threshold is colder than a predetermined minimum sensed temperature in the absence of aerosol and at a predetermined maximum flow rate.

18. The combination of claim 16, wherein the controller is constructed and arranged to vary the predetermined temperature threshold as a function of sensed fluid flow rate.

19. A method for detecting fluid flow past a flow sensor, the flow sensor comprising a base defining upstream and downstream directions, a heater disposed on the base, a first temperature sensor positioned so as to sense a first temperature at a first location, and a downstream temperature sensor disposed on the base downstream of the heater so as to sense a downstream temperature of the base downstream from the heater, the method comprising:
causing the heater to generate heat;
detecting, via the first temperature sensor, a first temperature at a first location on the base;
detecting, via the downstream temperature sensor, a downstream temperature of the base downstream from the heater; and
determining whether fluid is flowing past the flow sensor by determining whether a temperature differential between the first temperature and the downstream temperature increases.

20. The method of claim 19, further comprising recording in a memory the determination of whether fluid is flowing past the flow sensor.

21. The method of claim 19, wherein determining whether a temperature differential between the first temperature and the downstream temperature increases comprises subtracting a temperature signal from one of the first temperature sensor and the downstream temperature sensor from the other of the first temperature sensor and the downstream temperature sensor.

22. The method of claim 19, wherein determining whether a temperature differential between the first temperature and the downstream temperature increases comprises dividing a temperature signal from one of the first temperature sensor and the downstream temperature sensor by the other of the first temperature sensor and the downstream temperature sensor.

23. The method of claim 19, wherein the temperature differential is sensed in terms of a unit of measurement that is correlated to a temperature difference between the upstream and downstream temperatures.

24. The method of claim 19, further comprising determining from a magnitude of the temperature differential a magnitude of the flow rate of the fluid past the base.

25. The method of claim 19, further comprising determining a direction of flow past the flow sensor based on the temperature differential.

26. The method of claim 19, further comprising determining a direction and magnitude of flow past the flow sensor based on the temperature differential.

27. The method of claim 19, wherein the first temperature sensor comprises an upstream temperature sensor that is disposed on the base upstream of the heater so as to sense an upstream temperature of the base upstream from the heater.

28. The method of claim 27, further comprising determining a direction of flow past the flow sensor by comparing the upstream and downstream temperature signals, the direction of flow being based on a sign of the temperature differential.

29. The method of claim 27, wherein the upstream and downstream temperature sensors are positioned such that when the heater is turned on and there is no fluid flow over the base, the upstream and downstream temperatures are substantially identical.

30. The method of claim 27, wherein:
the base comprises a frame and a membrane connected to the frame,
the frame has a higher thermal capacitance than the membrane,
the heater is disposed on the membrane,
the downstream temperature sensor is positioned to sense a temperature of the membrane downstream from the heater, and
the upstream temperature sensor is positioned to sense a temperature of the membrane upstream from the heater.

31. The method of claim 27, wherein:
the base comprises a silicon frame and a membrane connected to the silicon frame,
the silicon frame has a higher thermal capacitance than the membrane,
the heater is disposed on the membrane,
the downstream temperature sensor comprises a thermocouple having a reference junction disposed on the silicon frame and a sensing junction disposed on the membrane downstream from the heater, and
the upstream temperature sensor comprises a thermocouple having a reference junction disposed on the silicon frame and a sensing junction disposed on the membrane upstream from the heater.

32. The method of claim 27, wherein:
the sensor is in thermal communication with a fluid pathway of an aerosol delivery system, the aerosol delivery system comprising an aerosol generator, and an aerosol output opening,
the fluid pathway extends to the aerosol output opening, the aerosol generator being positioned such that aerosol generated by the aerosol generator enters the fluid pathway, and
the downstream direction of the base is directed along the fluid pathway toward the aerosol output opening.

33. The method of claim 32, further comprising determining a direction and magnitude of flow in the fluid pathway based on the temperature differential.

34. The method of claim 32, further comprising detecting the presence of aerosol in the fluid pathway from at least one of the first and downstream temperatures.
